(19) United States
(12) Patent Application Publication
(10) Pub. No.: US 2020/0390793 A1
(43) Pub. Date: Dec. 17, 2020

(54) COMPOSITIONS AND METHODS USING OLEUROPEIN OR CURCUMIN FOR MUSCLE QUALITY AND/OR MUSCLE MASS

(71) Applicant: SOCIETE DES PRODUITS NESTLE S.A., Vevey (CH)

(72) Inventors: Claire Boutry, Villars Burquin (CH); Denis Breuille, Lausanne (CH); Marie Noelle Horcajada, Echenevex (FR)

(21) Appl. No.: 16/764,260

(22) PCT Filed: Nov. 20, 2018
(86) PCT No.: PCT/EP2018/081830

§ 371 Date: May 14, 2020

Related U.S. Application Data
(60) Provisional application No. 62/589,208, filed on Nov. 21, 2017.

(51) Int. Cl. A61K 31/7048; A23L 33/105; A61K 31/12; A61P 21/00

(52) U.S. Cl. CPC A61K 31/7048; A23L 33/105; A23V 2002/00; A61P 21/00; A61K 31/12

(57) ABSTRACT

A composition comprising at least one of oleuropein or curcumin can treat or prevent sarcopenia, reduce a loss of at least one of muscle quality or muscle mass, increase at least one of muscle quality or muscle mass, and/or improve recovery of at least one of muscle quality or muscle mass after muscle atrophy. The composition can be administered to an individual who is elderly and/or frail.

### BACKGROUND

The present disclosure generally relates to compositions and methods which use at least one of oleuropein or curcumin to improve or maintain at least one of muscle quality or muscle mass. More specifically, the present disclosure relates to administering a composition comprising at least one of oleuropein or curcumin to treat, prevent or reduce the progression of sarcopenia; reduce a loss of at least one of muscle quality or muscle mass; increase at least one of muscle quality or muscle mass; and/or improve recovery of at least one of muscle quality or muscle mass after muscle atrophy.

Sarcopenia is defined as a condition characterized by a progressive and generalized loss of skeletal muscle mass and function. One of the causes for this decline is the change in the quality of the muscle (fibers size and number). Skeletal muscle contraction time, strength and resistance to fatigue highly depend on the fiber type distribution and the degree of expression of the different myosin-heavy chain isoforms in the muscle.

Effective measures to preserve or improve muscle quality remain lacking.

### SUMMARY

In a general embodiment, the present disclosure provides a method of reducing a loss of at least one of muscle quality or muscle mass in an individual, increasing at least one of muscle quality or muscle mass, and/or improving recovery of at least one of muscle quality or muscle mass after muscle atrophy in an individual. The method comprises administering a composition comprising at least one of oleuropein or curcumin to the individual.

In an embodiment, the composition is administered at least twice a week for a time period of at least one month.

In an embodiment, the individual has sarcopenia.

In an embodiment, the method comprises, prior to the administering of the composition, identifying the individual as being in need of reduced loss of at least one of muscle quality or muscle mass in an individual, increased muscle quality and/or muscle mass, and/or improved recovery of at least one of muscle quality or muscle mass after muscle atrophy.

In another embodiment, the present disclosure provides a composition comprising at least one of oleuropein or curcumin in an amount that is therapeutically effective for at least one of: (i) reducing a loss of at least one of muscle quality or muscle mass in an individual, (iv) increasing at least one of muscle quality or muscle mass in an individual, or (v) improving recovery of at least one of muscle quality or muscle mass after muscle atrophy in an individual.

In an embodiment, the composition is selected from the group consisting of food compositions, dietary supplements, nutritional compositions, nutraceuticals, powdered nutritional products to be reconstituted in water or milk before consumption, food additives, medicaments, drinks, and combinations thereof.

In another embodiment, the present disclosure provides a method of making a food composition. The method comprises adding at least one of oleuropein or curcumin to another ingredient to form the food composition, the polyphenol added in an amount therapeutically effective to reduce a loss of at least one of muscle quality or muscle mass in an individual, increase at least one of muscle quality or muscle mass in an individual, and/or improve recovery of at least one of muscle quality or muscle mass after muscle atrophy in an individual.

An advantage of the present disclosure is to provide a composition, such as a food product or a food supplement, that treats sarcopenia in individuals.

Another advantage of the present disclosure is to provide a composition, such as a food product or a food supplement, that prevents sarcopenia.

Still another advantage of the present disclosure is to provide a composition, such as a food product or a food supplement, that reduces a loss of at least one of muscle quality or muscle mass in individuals, relative to the loss that would be experienced during consumption of a diet lacking the composition.

An additional advantage of the present disclosure is to provide a composition, such as a food product or a food supplement, that increases at least one of muscle quality or muscle mass in individuals, relative to that which would be present from consumption of a diet lacking the composition.

Another advantage of the present disclosure is to provide a composition, such as a food product or a food supplement, that improves recovery of at least one of muscle quality or muscle mass after muscle atrophy in individuals, relative to the recovery that would be present from consumption of a diet lacking the composition.

Yet another advantage of the present disclosure is to beneficially promote reduction, prevention, or treatment of sarcopenia in individuals.

Another advantage of the present disclosure is to provide nutritional strategies to reduce development of sarcopenia in individuals, especially to reduce loss of muscle quality and/or muscle mass in elderly humans.

Additional features and advantages are described in, and will be apparent from, the following Detailed Description and the Figures.

### BRIEF DESCRIPTION OF DRAWINGS

FIG. 1 shows the effect of age on skeletal muscles weight (mass) normalized to body weight for various muscles in adult rats, early sarcopenic rats, and sarcopenic rats.

FIG. 2 shows the effect of age on tibialis anterior muscle fiber size distribution in adult rats, early sarcopenic rats, and sarcopenic rats.

FIG. 3 shows effect of curcumin (CUR), rutin (RUT) and oleuropein (OLE) on correlation between muscle mass vs. fiber cell area (FIG. 3A) and percentage of cells with an area below 700 μm² (FIG. 3B) in old rats. 20 months-old rats were fed either a control diet or the same diet supplemented with curcumin or rutin or oleuropein for 3 months. CON=control diet, RUT=control diet with rutin, OLE=control diet with oleuropein and CUR=control diet with curcumin.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment comprises more than 50 wt. % of the identified components, preferably at least 75 wt. % of the identified components, more preferably at least 85 wt. % of the identified components, most preferably at least 95 wt. % of the identified components, for example at least 99 wt. % of the identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to, rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “patient” is understood to include an animal, especially a mammal, and more especially a human that is receiving or intended to receive treatment, as treatment is herein defined. While the terms “individual” and “patient” are often used herein to refer to a human, the present disclosure is not so limited. Accordingly, the terms “individual” and “patient” refer to any animal, mammal or human that can benefit from the treatment.

The term “elderly” means a person above the age of 60 years, preferably above 63 years, and more preferably above 65 years. The term “frail” refers to a person which is physically weak, i.e. not strong, but fragile.

The terms “treatment” and “treating” include any effect that results in the improvement of the condition or disorder, for example lessening, reducing, modulating, or eliminating the condition or disorder. The term does not necessarily imply that a subject is treated until total recovery. Non-limiting examples of “treating” or “treatment of” a condition or disorder include: (1) inhibiting the condition or disorder, i.e. arresting the development of the condition or disorder or its clinical symptoms and (2) relieving the condition or disorder, i.e. causing the temporary or permanent regression of the condition or disorder or its clinical symptoms. A treatment can be patient- or doctor-related.

The terms “prevention” or “preventing” mean causing the clinical symptoms of the referenced condition or disorder to not develop in an individual that may be exposed or predisposed to the condition or disorder but does not yet experience or display symptoms of the condition or disorder. The terms “condition” and “disorder” mean any disease, condition, symptom, or indication.

The relative terms “improved,” “increased,” “enhanced” and the like refer to the effects of the composition comprising at least one of oleuropein or curcumin (disclosed herein) relative to a composition lacking the at least one of oleuropein or curcumin but otherwise identical.

The terms “food,” “food product” and “food composition” mean a product or composition that is intended for ingestion by an individual such as a human and provides at least one nutrient to the individual. The compositions of the present disclosure, including the many embodiments described herein, can comprise, consist of, or consist essentially of the essential elements and limitations described herein, as well as any additional or optional ingredients, components, or limitations described herein or otherwise useful in a diet.

As used herein, “complete nutrition” contains sufficient types and levels of macronutrients (protein, fats and carbohydrates) and micronutrients to be sufficient to be a sole source of nutrition for the animal to which the composition is administered. Individuals can receive 100% of their nutritional requirements from such complete nutritional compositions.

As used herein, “muscle quality” is the volume or area per muscle cell, e.g., the total volume or total area of a plurality of muscle cells, divided by the number of muscle cells. Muscle quality is finally correlated with muscle mass and ultimately to sarcopenia as above. Sarcopenia is a combination of loss of muscle functionality and loss of muscle quality.

### Embodiments

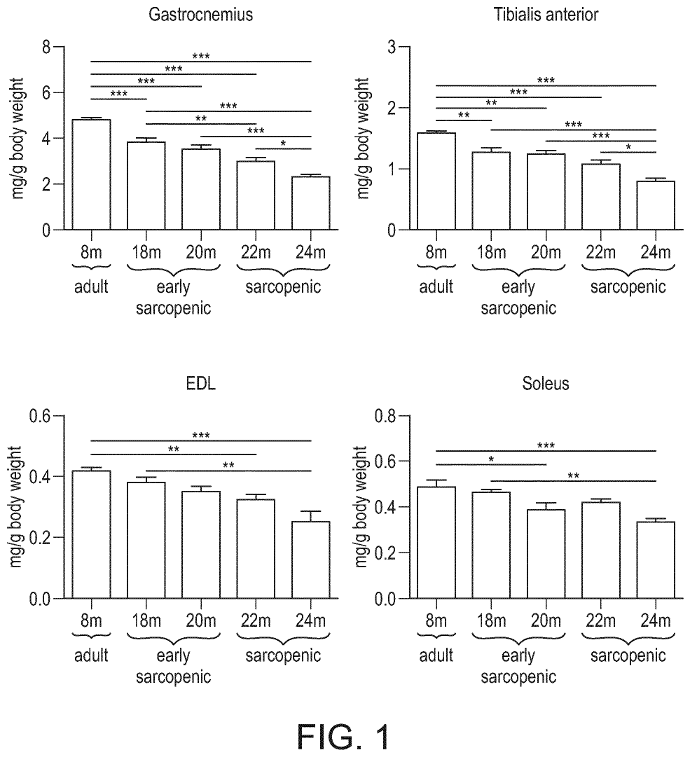

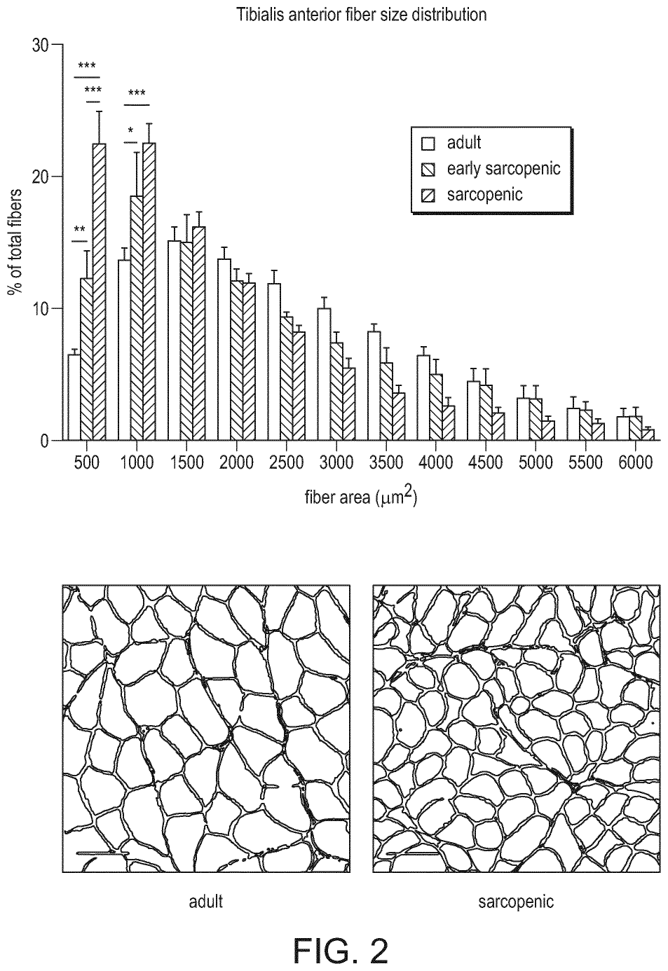

Applicant investigated muscle quality in adult rats, early sarcopenic rats, and sarcopenic rats. FIG. 1 shows that muscle mass decreases with sarcopenia. FIG. 2 shows that muscle fiber crossectional area decreases with age and with sarcopenia. There is a higher percentage of small fibers area and a lower percentage of big fibers area.

Accordingly, an aspect of the present disclosure is a composition comprising at least one of oleuropein or curcumin for treatment or prevention of sarcopenia, for reducing a loss of at least one of muscle quality or muscle mass, for increasing at least one of muscle quality or muscle mass, and/or for improving recovery of at least one of muscle quality or muscle mass after muscle atrophy in an individual such as a mammal, e.g., a human.

Another aspect of the present disclosure is a method comprising administering a therapeutically effective amount of a composition comprising at least one of oleuropein or curcumin to an individual to treat the individual for sarcopenia, prevent sarcopenia in the individual, reduce a loss of at least one of muscle quality or muscle mass in the individual, increase at least one of muscle quality or muscle mass in the individual, and/or improve recovery of at least one of muscle quality or muscle mass after muscle atrophy in the individual. The method can comprise identifying, preferably prior to the administering of the composition, the individual as having a characteristic selected from the group consisting of (i) in need of reduced loss of muscle quality and/or muscle mass, (ii) in need of improved muscle quality and/or muscle mass and (iii) in need of improved recovery of muscle quality and/or muscle mass after muscle atrophy.

In an embodiment, the composition comprises an amount of the oleuropein and/or curcumin of from 0.01 mg to about 1 g, preferably from 0.1 mg to 1 g, more preferably from 1 mg to about 1 g, most preferably from 40 mg to 500 mg per serving. For example, an amount of the oleuropein and/or curcumin administered to the individual can be 100 mg to 300 mg per day. A non-limiting example of an embodiment comprises administration of about 120 mg of the oleuropein per day and/or 250 mg of the curcumin per day. The daily dose can be provided by one or more servings of the composition per day, preferably in one or two doses per day.

Muscle atrophy, as treated or prevented according to the present disclosure, may be caused by many reasons. For example, it may result from lack of physical activity, such as from immobilization or low physical activity associated with aging (sarcopenia associated with aging process), hip-fracture recovery, or several co-morbidities of diseases, such as cancer, AIDS, congestive heart failure, COPD (chronic obstructive pulmonary disease), renal failure, trauma, sepsis, and severe burns, for example. Muscle atrophy may result from insufficient or inappropriate nutrition or starvation. Very commonly, muscle atrophy results from disuse or insufficient use of the respective muscle and/or from metabolic changes. In some embodiments, the improved or maintained muscle quality and/or muscle mass are achieved without modification of muscle functionality (e.g., gait speed and/or total muscle strength).

The muscle referenced in the present disclosure is preferably a skeletal muscle. For example, the composition disclosed herein may be used to reduce the loss of at least one of muscle quality or muscle mass in the arms and/or the legs of the individual. The muscle may be one or more of the following: gastrocnemius, tibialis, soleus, extensor, digitorum longus (EDL), biceps femoris, semitendinosus, semimembranosus, or gluteus maximus.

Muscle atrophy may result in the disorder of sarcopenia, i.e., lost muscle mass, size, and functionality because of aging. The muscle atrophy may be of different grades, such as severe muscle atrophy as in extreme frailty of elderly persons. Extremely frail elderly persons can have difficulty in every-day activities and taking care of themselves. Muscle atrophy of a less severe degree will allow some movement and some muscle activity, but the muscle activity is insufficient to sustain the complete muscle tissue. The mechanisms involved in treating or preventing age-associated sarcopenia are different from treating or preventing loss of muscle quality or muscle mass in younger persons.

The composition disclosed herein comprises at least one of oleuropein or curcumin and can reduce loss of at least one of muscle quality or muscle mass and/or improve at least one of muscle quality or musclde mass in an individual who is administered the composition, relative to a diet lacking such a composition. In a preferred embodiment, the oleuropein and/or curcumin are present in food-grade material. A material is considered “food-grade” if it is generally accepted and considered safe for food applications.

At least a portion of the oleuropein and/or curcumin may be a purified compound or a partially purified compound. At least a portion of the oleuropein and/or curcumin may be provided by one or more plant, algal or fungal extracts or fractions rich in oleuropein and/or curcumin respectively. In an embodiment, the oleuropein and/or curcumin may be the only polyphenols in the composition.

In addition to the oleuropein and/or curcumin, the composition can further comprise a protein source from animal or plant origin, for example milk proteins, soy proteins, and/or pea proteins. In a preferred embodiment, the protein source is selected from the group consisting of whey protein; casein protein; pea protein; soy protein; wheat protein; corn protein; rice protein; proteins from legumes, cereals and grains; and combinations thereof. Additionally or alternatively, the protein source may comprise a protein from nuts and/or seeds.

The protein source preferably comprises whey protein. The whey protein may be unhydrolyzed or hydrolyzed whey protein. The whey protein may be any whey protein, for example the whey protein can be selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof. In a preferred embodiment, the whey protein comprises whey protein isolate and/or modified sweet whey.

As noted above, the protein source can be from animal or plant origin, for example milk proteins, soy proteins, and/or pea proteins. In an embodiment, the protein source comprises casein. Casein may be obtained from any mammal but is preferably obtained from cow milk and preferably as micellar casein.

The composition can comprise one or more branched chain amino acids. For example, the composition can comprise leucine, isoleucine and/or valine. The protein source in the composition may comprise leucine in free form and/or leucine bound as peptides and/or proteins such as dairy, animal or vegetable proteins. In an embodiment, the composition comprises the leucine in an amount up to 10 wt % of the dry matter of the composition. Leucine can be present as D- or L-leucine and preferably the L-form. If the composition comprises leucine, the composition can be administered in a daily dose that provides 0.01 to 0.04 g of the leucine per kg body weight, preferably 0.02 to 0.035 g of the leucine per kg body weight. Such doses are particularly applicable to complete nutrition compositions, but one of ordinary skill will readily recognize how to adapt these doses for an oral nutritional supplement (ONS).

In addition to the oleuropein and/or curcumin, another antioxidant may be used in the composition. For example, these additional antioxidants may be provided as food compositions that are rich in antioxidants or as extracts thereof. A food composition that is “rich in antioxidants” has an ORAC (oxygen radical absorbance capacity) rating of at least 100 per 100 g of the composition.

The composition comprising oleuropein and/or curcumin can be administered to an individual such as a human, e.g., an elderly human, in a therapeutically effective dose. The therapeutically effective dose can be determined by the person skilled in the art and will depend on a number of factors known to those of skill in the art, such as the severity of the condition and the weight and general state of the individual.

The composition may be administered to an individual in an amount sufficient to prevent or at least partially reduce the risk of developing sarcopenia in instances where the condition of sarcopenia has yet not been developed in the individual. Such an amount is defined to be “a prophylactically effective dose.” Again, the precise amounts depend on a number of factors relating to the individual, such as their weight, health and how much muscle quality or muscle mass is being lost.

The composition is preferably administered as a supplement to the diet of an individual daily or at least twice a week. In an embodiment, the composition is administered to the individual consecutively for a number of days, preferably until an increase in at least one of muscle quality or muscle mass relative to that before administration is achieved. For example, the composition can be administered to the individual daily for at least 30, 60 or 90 consecutive days. As another example, the composition can be administered to the individual for a longer period, such as a period of 1, 2, 3, 4, 5, 6, 7, 8, 9 or 10 years.

In a preferred embodiment, the composition is administered to the individual for at least 3 months, for example a period of 3 months to 1 year, and preferably for at least 6 months.

The above examples of administration do not require continuous daily administration with no interruptions. Instead, there may be some short breaks in the administration, such as a break of two to four days during the period of administration. The ideal duration of the administration of the composition can be determined by those of skill in the art.

In a preferred embodiment, the composition is administered to the individual orally or enterally (e.g. tube feeding). For example, the composition can be administered to the individual as a beverage, a capsule, a tablet, a powder or a suspension.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, for example the oleuropein and/or curcumin separately from one or more of the other components of the composition, and/or or a portion of the oleuropein and/or curcumin separately from another portion of the oleuropein and/or curcumin.

In some embodiments, the compositions consist essentially of the oleuropein and/or curcumin and optionally one or more of a protein source, a free amino acid, a carbohydrate source, a fat source, a vitamin, or a mineral. In an embodiment, the composition comprising at least one of oleuropein or curcumin further comprises vitamin D.

### Example

The following non-limiting example presents scientific data developing and supporting the concept of administering a composition comprising oleuropein and/or curcumin to treat sarcopenia, prevent sarcopenia, reduce a loss of muscle quality and/or muscle mass, increase muscle quality and/or muscle mass, and/or improve recovery of muscle quality and/or muscle mass in an individual to whom the composition is administered.

Old rats are a good model to assess the effect of nutritional intervention in age related muscle decline. With this model, it can be observed as in human a decrease in muscle mass and function and a change in muscle quality with age. The objective of the study was to determine the effects of curcumin, oleuropein and rutin on muscle quality in old rats and the association with muscle mass.

20 months-old rats have been divided in four groups and received a complete diet supplemented with either curcumin (CUR), oleuropein (OLE) or rutin (RUT) for 3 months. A control group (CON) received cellulose instead of polyphenols. Fiber type was analyzed in muscle by immunofluorescence staining and microscopy. Exact Wilcoxon test associated with Hodges-Lehmann to estimate the difference between groups and Kolmogorov-Smirnov test to assess the difference for the distribution have been performed for statistical analysis.

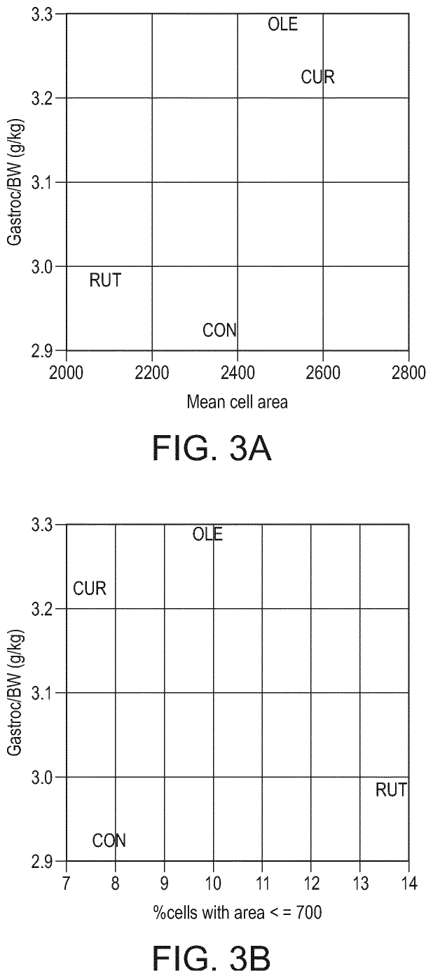

The results (e.g., the histograms in FIGS. 3A and 3B) showed that CUR and OLE groups exhibited higher muscle mass, higher fiber crossectionnal area (FIG. 3A) and lower amount of small fibers area (FIG. 3B) than control and rutin groups.

In conclusion, dietary oleuropein and/or curcumin supplementation can influence the quality of the muscle and could be a solution to tackle one of the causes of age related muscle decline. Rutin supplementation did not show this effect.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of achieving a result selected from the group consisting of reducing a loss of at least one of muscle quality or muscle mass in an individual, increasing at least one of muscle quality or muscle mass, and improving recovery of at least one of muscle quality or muscle mass after muscle atrophy in an individual, the method comprising administering a composition comprising at least one of oleuropein or curcumin to the individual.

2. The method of claim 1 wherein the composition is administered at least twice a week for a time period of at least one month.

3. The method of claim 1 wherein the individual has sarcopenia.

4. The method of claim 1 comprising, prior to the administering of the composition, identifying the individual as being in need of reduced loss of at least one of muscle quality or muscle mass in an individual, increased muscle quality and/or muscle mass, and/or improved recovery of at least one of muscle quality or muscle mass after muscle atrophy.

5. A composition comprising at least one of oleuropein or curcumin in an amount that is therapeutically effective for at least one of: (i) reducing a loss of at least one of muscle quality or muscle mass in an individual, (iv) increasing at least one of muscle quality or muscle mass in an individual, and (v) improving recovery of at least one of muscle quality or muscle mass after muscle atrophy in an individual.

6. The composition of claim 5 wherein the composition is selected from the group consisting of food compositions, dietary supplements, nutritional compositions, nutraceuticals, powdered nutritional products to be reconstituted in water or milk before consumption, food additives, medicaments, drinks, and combinations thereof.

7. A method of making a food composition, the method comprising adding at least one of oleuropein or curcumin to another ingredient to form the food composition, the polyphenol added in an amount therapeutically effective to achieving a result selected from the group consisting of reduce a loss of at least one of muscle quality or muscle mass in an individual, increase at least one of muscle quality or muscle mass in an individual, and/or improve recovery of at least one of muscle quality or muscle mass after muscle atrophy in an individual.
